(19) United States
(12) Patent Application Publication
(10) Pub. No.: US 2012/0087980 A1
(43) Pub. Date: Apr. 12, 2012

(54) STIMULATION OF NEUROREGENERATION BY FLAVONOID GLYCOSIDES

(76) Inventor: Marcy Zenobi-Wong, Zurich (CH)

(21) Appl. No.: 13/250,844

(22) Filed: Sep. 30, 2011

Related U.S. Application Data
(60) Provisional application No. 61/391,018, filed on Oct. 7, 2010.

(51) Int. Cl. A61K 9/00; A61P 25/00; A61P 25/28; C07H 7/06; A61K 35/12; A61K 35/30; C07H 15/26; C07H 17/07; A61K 31/7048; A61K 38/45

(52) U.S. Cl. 424/484; 514/24; 514/23; 514/25; 514/27; 514/42; 424/94.5; 424/93.7; 536/8; 536/29.1; 536/1.11

(57) ABSTRACT

Flavonoid glycosides, such as isoquercitrin, are shown to stimulate the formation of neuritis and neuronal synapses in neurons and neuronal progenitor (stem) cells.

### CROSS REFERENCE TO RELATED APPLICATION

This application claims priority from U.S. Provisional Application Ser. No. 61/391,018, filed Oct. 7, 2010, the contents of which are incorporated herein by reference.

### BACKGROUND DISCUSSION

Flavonoids belong to a group of polyphenols with putative anti-oxidant, anti-cancer and anti-aging properties. These molecules are naturally occurring in fruits and vegetables and are orally active. One flavonoid class, flavonols (3-hydroxy-2-phenylchromen-4-one) are particularly relevant to applications in neuroregeneration. Fisetin, that is a flavonol aglycone, has been reported to induce neurite outgrowth in PC 12 cells via the ERK pathway [1] and may enhance memory and learning.

As certain flavonoids are known to inhibit the RhoA/Rho Kinase pathway [2] and Rho GTPases act as molecular switches controlling cytoskeletal changes of neuritogenesis, cell polarization and synapse formation, we investigated the activation state of RhoA as well as the gene expression profile of members of the Rho GTPase family upon incubation with flavonol glycosides. Cells with polarized, elongated morphology have been described in the literature [3, 4]. The degree of elongation appears to depend on the efficiency of disassembly of adhesions which are the rear of the migrating will, with both Src and Rho GTPases playing an important role. While RhoA activation induces tail release [5] and a more rounded cell morphology, the inhibition of its downstream effector ROCK by Y-27632 is associated with an elongated morphology in PC3 human prostatic cancer cells [6], NIH 3T3 fibroblasts [7], and podocytes [8]. In neurons, RhoA activation caused neurite retraction and growth cone release [9, 10] while suppression of p160ROCK induced neuronal morphology in N1E-115 neuroblastoma cells [11] and potentiated NGF-induced neurite outgrowth in PC12 cells [12]. Similarly Rho inhibitors induced formation of dendritic processes in melanocytes, while a Rho activator blocked the forskolin induced formation of dendrites [13]. Y-27632 may also stabilize the tubulin cytoskeleton [8] and has been reported to support neuronal survival in injury and neurological disorders [14].

### SUMMARY OF THE INVENTION

We have found that certain flavonoid glycosides, such as isoquercitrin, when administered to a human or animal in a therapeutically effective amount stimulate the formation of neurites and neuronal synapses in neurons and neuronal progenitor (stem) cells. Furthermore, the combination of flavonoid glycoside treatment with inhibitors of RhoA such as Y-27632, Fasudil, and Exoenzyme C3 Transferase further augments neurite formation and protects nerve cells from excitotoxic effects and oxidative damage. The potent effect on neuroregeneration advantageously may be used in the treatment of spinal cord and peripheral nerve injuries, stroke, as well as neurological and cognitive disorders such as Alzheimers, Parkinsons, Amyotrophic Lateral Sclerosis (ALS), and optical nerve atrophy (Leber's Disease). The substances also may be used to treat age-associated memory loss, learning disability, autism and dementia.

More particularly, we have found that pharmaceutical compositions comprising a therapeutically effective amount of a compound according to Formula I:

wherein each of R₅, R₆, R₇, R₈, R_{2′}, R_{3′}, R_{4′} and R_{5′} is independently H, OH or OCH₃; and wherein each of R₃, R₇, R₈ and R₄′ is independently a hydrogen atom or a monosaccharide bound to its respective ring structure by an O—, S—, N— or C— glycosidic bond through one of R₃, R₇, R₈ or R_{4′}, or a pharmaceutically-acceptable salt, enantiomer, diasteriomer, recemic mixture, enantiomerically-enriched mixture, solvate, or prodrug of a compound according to Formula I, in a pharmaceutically acceptable carrier, and which is present in or administered in a pharmaceutically-effective amount.

In one embodiment, the monosaccharide group may assume either a D or L chirality.

In a preferred embodiment, the monosaccharide is a glucoside, a glucorhamnoside, a galactoside, a glucuronide or a xyloside. Particularly effective flavored glucosides in promoting neurite growth are fisetin, kaempferol, and quercetin-3-β-D glycoside (isoquercitrin). All are available commercially.

### BRIEF DESCRIPTION OF THE DRAWINGS

Further features and advantages of the present invention will be seen through the following detailed description, taken in conjunction with the accompanying drawings, wherein:

FIGS. 1A-1D shows immunofluorescence analysis of NG108-15 cells. More particularly, FIGS. 1A-1D show Immunofluorescence analysis of NG108-15 cells grown on laminin coated wells. Upper left (FIG. 1A): Control (48 hrs culture); upper right (FIG. 1B): cells stimulated with 40 uM isoquercitrin; lower left (FIG. 1C): cells incubated with 40 uM isoquercitrin+10 uM Y-27632; lower right (FIG. 1D): cells incubated with 40 uM isoquercitrin+0.25 μg/ml C3 Transferase.

FIG. 2 comprises a time-lapse photograph showing isoquercitrin-stimulated NG108-15 cells. More particularly, FIG. 2 shows time lapse acquisition of an isoquercitrin-stimulated NG108-15 cell. The images, acquired every 56 minutes, show themigration path and neurite extension out of the cell body. Note that the neurite tail is undergoes a small amount of translation, where the top of each image is at the same position. Scale bar=50 uM.

FIGS. 3A and 3B show fluorescence of NG108-15 cells transfected with Far-Red-vinculin and stimulated with 40 μM isoquercitrin for 48 hours. More particularly, FIGS. 3A and 3B show fluorescence of NG108-15 cells transfected with Far Red-vinculin and stimulated with 40 uM isoquercitrin for 48 hrs. Vinculin rich focal adhesions were identified in the tips of the neurite processes.

FIG. 4 provides an analysis of neurite-length cell versus time measurements based on a 48 hour time-lapse image accessory. More particularly, FIG. 4 shows—Top: Analysis of neurite length/cell vs. time based on 48 hrs time-lapse image acquisition. NG108 control cells and NG108 cells stimulated with isoquercitrin (Q), isoquercitrin+C3 Transferase RhoA inhibitor, and isoquercitrin+ROCK kinase inhibitor Y27632. Bottom: Quantification of neurite retraction upon addition of Phospholipase C inhibitor U73122 or RhoA activator calpeptin.

FIGS. 5A and 5B are graphs showing gene expression analysis of Rho GT Pases upon 24 hour isoquercitrin stimulation. More particularly, FIGS. 5A and 5B show gene expression analysis of Rho GTPases upon 24 hr isoquercitrin stimulation. RhoA and Rac1 expression is down-regulated, while expression of RhoQ and RhoV is upregulated.

FIG. 6 illustrates RhoA activation. More particularly, FIG. 6 shows RhoA activation G-LISA. RhoA activity is depressed following administration of isoquercitrin for 48 hrs. As a control, incubation with RhoA Activator for 40 min after 48 hr isoquercitrin incubation increases RhoA activation.

### DETAILED DESCRIPTION OF PREFERRED EMBODIMENTS

As used in the present invention, to state that a composition “includes a compound according to (or of) a particular formula, or a pharmaceutically-acceptable salt, enantiomer, diastereomer, racemic mixture, enantiomerically-enriched mixture, solvate, or prodrug of such formula”, means that such composition may include either a single compound falling within such definition, or may include more than one compound falling within such definition. For example, a pharmaceutical composition of the present invention that includes a “therapeutically effective amount of a compound according to Formula I, or a pharmaceutically-acceptable salt, enantiomer, diasteriomer, racemic mixture, enantiomerically-enriched mixture, solvate, or prodrug of Formula I may include a single compound according to Formula I, or may include one compound that meets the above definition in combination with another combination that also meets the above definition, or another compound that does not meet the above definition. Thus, pharmaceutical compositions of the present invention may include any number of compounds in combination that fall within the provided definitions, so long as they are therapeutically or otherwise useful as intended.

Pharmaceutical compositions including compounds according to Formula I, or pharmaceutically-acceptable salts, enantiomers, diastereomers, solvates, or prodrugs of Formula I, may be administered to a human or animal patient in need thereof in any acceptable form in dosage unit formulations that employ conventional non-toxic pharmaceutically acceptable carriers, adjuvants and vehicles that permit such compositions of the present invention to have therapeutic activity. For example, such compositions may be in an orally-administrable form; a topical form; may be administered to the sinuses, throat or lungs; or may be administered parenterally, rectally or vaginally.

Pharmaceutical compositions of the present invention that are intended for oral use may be in the form of a pill, tablet, gelcap, or hard or soft capsule (each of which may be in an immediate, sustained or time-release formulation); lozenge; throat spray; solution; emulsion; cream; paste; gel; cough drop; dissolvable strip; lollipop; gum; aqueous or oily suspension, dispersible powder/granules; syrup or elixir; and may be prepared according to any method known in the art for the manufacture of pharmaceutical compositions. Such compositions may further contain one or more agents selected from the group consisting of sweetening agents, flavoring agents, coloring agents and preserving agents. By dissolvable strip is meant a sheet of material that can be placed in the mouth to dissolve and release the active ingredient or prodrug. Such dissolvable strips are also known as flavor strips or oral care strips. Dissolvable strips are often carbohydrate-based. Tablets typically contain the active ingredient or prodrug in admixture with non-toxic pharmaceutically acceptable excipients which are suitable for the manufacture of such tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example, magnesium stearate, stearic acid or talc. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

With respect to capsules, in hard gelatin capsule formulations the active ingredient(s) or prodrug may be mixed with an inert solid diluent, for example, calcium carbonate, calcium phosphate or kaolin; and in soft gelatin capsule formulations the active ingredient(s) or prodrug may be mixed with water or an oil medium, for example peanut oil, liquid paraffin, or olive oil.

Aqueous suspensions typically contain the active material in admixture with excipients suitable for the manufacture of such aqueous suspensions. Such excipients may be suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents, such as a naturally-occurring phosphatide (e.g., lecithin), condensation products of an alkylene oxide with fatty acids (e.g., polyoxyethylene stearate), condensation products of ethylene oxide with long chain aliphatic alcohols (e.g., heptadecaethylene-oxycetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives, for example ethyl, n-propyl, or p-hydroxybenzoate; one or more coloring agents; one or more flavoring agents; and/or one or more sweetening agents, such as sucrose, saccharin or aspartame.

Oily suspensions may be formulated by suspending the active ingredient or prodrug in a vegetable oil, such as arachis oil, olive oil, sesame oil or coconut oil; or in mineral oil, such as liquid paraffin. Such oily suspensions may also contain a thickening agent, such as beeswax, hard paraffin or cetyl alcohol. Sweetening agents, such as those set forth above, and flavoring agents, may also be added to provide a palatable oral preparation. Such compositions may be preserved by the addition of an anti-oxidant, such as ascorbic acid.

Dispersible powders and granules suitable for preparation of an aqueous suspension by the addition of water typically provide the active ingredient or prodrug in admixture with a dispersing or wetting agent, a suspending agent and one or more preservatives. Suitable dispersing or wetting agents and suspending agents are exemplified by those aforementioned. Additional excipients, for example sweetening, flavoring and coloring agents, may also be present.

Pharmaceutical compositions of the present invention may also be in the form of oil-in-water emulsions. In such preparations, the oily phase may be a vegetable oil (e.g., olive oil or arachis oil), a mineral oil (e.g., liquid paraffin), or mixtures of such vegetable and mineral oils. Suitable emulsifying agents may be naturally-occurring phosphatides, such as soy bean, lecithin, esters or partial esters derived from fatty acids and hexitol anhydrides (e.g., sorbitan monooleate), and condensation products of such partial esters with ethylene oxide (e.g., polyoxyethylene sorbitan monooleate). Such emulsions may also contain sweetening and flavoring agents.

Syrups and elixirs may be formulated with sweetening agents, such as glycerol, propylene glycol, sorbitol or sucrose. Such formulations may also contain a demulcent, a preservative, and flavoring and/or coloring agents. Pharmaceutical compositions of the present invention may also be in the form of a sterile injectable aqueous or oleaginous suspension, which may be formulated according to methods known in the art using, for example, suitable aforementioned dispersing or wetting agents, and suspending agents. Such a sterile injectable preparation may also be a sterile injectable solution or suspension in a non-toxic parenterally-acceptable diluent or solvent, such as a solution in 1,3-butane diol. Acceptable vehicles and solvents that may be employed include, for example, water, Ringer's solution and isotonic sodium chloride solution. Additionally, sterile, fixed oils may be employed as a solvent or suspending medium, such as a bland fixed oil, including synthetic mono- or diglycerides. Fatty acids such as oleic acid, may also be used in the preparation of injectables.

As aforementioned pharmaceutical compositions of the present invention may be administered in a controlled or sustained release system. Such systems include, for example, the use of a pump (see, for example, Langer and Sefton, (1987) CRC Crit. Ref. Biomed. 14:201: Buchwald et al. (1980), Surgery 88:507; Saudek et al. (1989), N. Engl. J. Med. 321:574), and more typically (with respect to oral formulations such as pills, tablets, etc.), the use of polymeric materials (see, for example, Medical Applications of Controlled Release, Langer and Wise (eds.) (1974), CRC Pres., Boca Raton, Fla.; Controlled Drug Bioavailability, Drug Product Design and Performance, Smolen and Ball (eds.) (1984), Wiley, New York; Ranger and Peppas, J. (1983), Macromol. Sc. Rev. Macromol. Chem. 23:61; Levy et al. (1985), Science 228:190; During et al. (1989), Ann. Neurol. 25:351; Howard et al. (1989), J. Neurosurg. 71:105). Other means of effecting controlled release involve, for example, placing the therapeutic composition in proximity of the therapeutic target, thus requiring only a fraction of the systemic dose (see, for example, Goodson, Medical Applications of Controlled Release, (1984) vol. 2, pp. 115-138). Other controlled release systems which may be employed include those reviewed by Langer (Science (1990) 249:1527-1533).

Pharmaceutical compositions including compounds of Formula I, or pharmaceutically-acceptable salts, enantiomers, diasteriomers, racemic mixtures, enantiomerically-enriched mixtures, solvates, or prodrugs of Formula I, also may be administered in the form of rectal or vaginal suppositories. Such compositions may be prepared by mixing the active compound with a suitable non-irritating excipient which is solid at ordinary temperatures but liquid at body temperature, and will therefore melt in the rectum or vagina to release the active compound. Suitable rectal and vaginal suppository materials include cocoa butter and polyethylene glycol.

For topical administration of the inventive compositions and compounds, a liquid solution; liquid spray; emulsion; cream; paste; gel; lotion; foam; impregnated dressing; ointment; jelly; mouth wash/gargle may be employed.

Pharmaceutical compositions of the present invention also may be administered occularly, such as in the form of eye-drops, ointments, sprays or conjunctival timed-release inserts. Administration of the inventive pharmaceutical compositions to the sinuses, throat, or lungs may be in the form of inhalable particles, inhalable solution, droplets, inhalation sprays, or aerosols. Further, such compositions may be administered parenterally, such as by subcutaneous injection, intravenously, intramuscularly, intrasternally, or by various infusion techniques.

The dosage regimen for pharmaceutical compositions of the present invention will, of course, vary depending upon known factors, such as the pharmacodynamic characteristics of the particular agent and its mode and route of administration; the species, age, sex, general health, medical condition, diet, and body weight of the recipient; the nature and extent of the symptoms; the kind of concurrent treatment; the frequency of treatment; the time of administration; route of administration, the renal and hepatic function of the patient, and the effect desired. A physician (or veterinarian in such case as the inventive compositions are use in the treatment of non-human animals) can determine and prescribe the effective amount of the drug required to prevent, counter, or arrest the progress of the disease state.

Moreover, the amount of active ingredient or prodrug that may be combined with carrier materials to produce a single dosage form will vary depending upon the host treated and the particular mode of administration. For example, a formulation intended for the oral administration of humans may contain from 0.05 mg to 500 mg per day of an active agent or prodrug compounded with an appropriate and convenient amount of carrier material which may vary from about 0.001 to about 0.05 percent of the total composition. Dosage unit forms will generally contain between from about 0.05 mg to about 50 mg of an active ingredient or prodrug, typically 0.05 mg, 0.1 mg, 1 mg, 5 mg, or 10 mg, or on an as-needed basis.

The present invention provides pharmaceutical compositions containing certain flavonoid glycoside-type compounds of Formula that have been shown to be therapeutically useful to stimulate neurogenesis in cells and tissues, and to methods for stimulating neurogenesis in cells and tissues based on the administration of such pharmaceutical compositions and compounds in the treatment and management of various injuries and disease in cells and tissues for patients suffering spinal cord or peripheral nerve injuries, stroke as well as neurological and cognitive disorders in humans and animals, such as Alzheimers, Parkinsons, ALS and Leber's Disease, or to treat age-associated memory loss, learning disability, autism and dementia.

Turning now to the non-limiting examples, the invention will be further illustrated.

### Preparation

### Materials and Methods:

Cell culture. The NG108-15 neuroblastoma/glioma cells and ND7/23 rat DRG/mouse neuroblastoma cells were seeded at 10⁴ cells/cm² on laminin coated wells and stimulated for 48 hours with 40 μM isoquercitrin (Cat No 00140585, Sigma) alone or in combination with either 10 μM Rho Kinase inhibitor Y-27632 (Cat No Y0503, Sigma) or 0.25 μg/ml Rho inhibitor Exoenzyme C3 Transferase (Cat No CT04, Cytoskeleton). Later, isoquercitrin stimulated cells were incubated with 0.2 unit/ml Rho Activator Calpeptin (Cat No CN01, Cytoskeleton) or, alternatively with 0.25 μM Phospholipase C inhibitor U73122 (Cat No 662035, Calbiochem).
Live Cell Imaging. During incubation, cells were observed by time-lapse live cell imaging with Cell-IQ (Chip-Man Technologies, Ltd). Quantitative analysis of neurite length/cell was performed using Cell-IQ Analyser Software.
Cell imaging by Immunofluorescence. Cells were cultured for 48 hrs on a Chamber Slide™ System (Lab-Tek™) under the above described conditions. Then, the cells were fixed and permeabilized with 4% formaldehyde/0.1% Triton X100 in PBS for 20 min at 4° C. After blocking with PBS containing 20% FBS and 5% BSA for 1 h at room temperature, they were incubated with mAb anti-synaptotagmin-1 (1:200, SY Synaptic Systems) for 1 hr at room temperature. Following three washes in PBS, cells were incubated with Alexa Fluor® 488 goat anti-mouse IgG/IgM (Invitrogen) for 30 min at room temperature. Afterwards, cells were incubated with 0.13 μg/ml phalloidin in the dark for 30 min at 4° C. Finally DAPI was added and last 30 min. Samples were rinsed three times and kept in PBS. The imaging was performed using Confocal Laser Scanning Microscope (Carl Zeiss AG).
Assay for RhoA activation. For RhoA activation assay, the procedure followed the manufacture's instruction regarding RhoA G-LISA™ Activation Assay (Cytoskeleton). Briefly, grown cells were homogenized in a lysis buffer and were centrifuged at 14,000 rpm for 2 min. The supernatants, containing equal amount of proteins (60 μg/30 μl), were transferred into a 96 well plate and equal volumes of ice-cold binding buffer were added into each well. The plate was incubated for 30 min under shaking (400 rpm) at 4° C. Then they were incubated with diluted anti-RhoA primary antibodies followed by secondary antibodies while shaking (400 rpm) at room temperature for 45 min each. The plate was incubated with an HRP detection reagent for 15 min at 37° C. and, after addition of an HRP stop buffer, the absorbance was immediately recorded at 490 nm.
Microarray and RT-PCR. Total cellular RNA was isolated using the RNAeasy spin columns with the DNAse digestion step. The purity and concentration were determined using a Nanoprop ND 1000 (Nanoprop Technologies, Delaware, USA) and a Bioanalyzer 2100 (Agilent, Waldbronn, Germany). All samples had 260/280 nm ratio above 1.8 and a 28S/18S ratio within 1.5-2. RNA (600 ng) was reverse transcribed into double-stranded complementary DNA (ds-cDNA) in the presence of RNA poly-A controls, RNA Spike-In Kit, Two-Color. The ds-cDNA was in vitro transcribed in the presence of Cy3 (control) and Cy5 (flavonoid-stimulated) labelled nucleotides using a Quick Amp labelling, two-color kit (Agilent P/N 5190-0444, Waldbronn, Germany). Transcription products (1.65 μg) were blocked, fragmented and hybridized to whole Mouse Genome 4×44k OligoMicroarrays (Agilent G4122F) for 17 h at 65° C. Arrays were then washed according to the manufacturer instructions and an Agilent Microarray Scanner used to measure the fluorescent intensity emitted by the labeled target. Total RNA was also reverse transcribed using oligo-dT primer and Superscript III enzyme (Invitrogen). The cDNA sequences corresponding to Gapdh (Fwd:, Rev:), RhoA (Fwd: GGG CGT GGA TGC GTT CT, Rev: ACG CGC GCA CAC TCT CA), Rac1 (Fwd: GCT AAC GCT GTC CTG TAC AAC CT, Rev: TGG TTG AAA GGC CCA ACA CT), Cdc42 (Fwd: CCC TCA CAC AGA AAG GCC TAA A, Rev: GCT CCA GGG CAG CCA AT), and Tc10 (Fwd: GCGCGTCCTGTGGGATT, Rev: GCTCCAAGCGGACATCAGTT) were amplified in triplicate by Real Time RT-PCR, using a StepOnePlus thermocycler with Fast SYBR Green Master Mix (Applied Biosystems).

### EXAMPLES

### Example 1

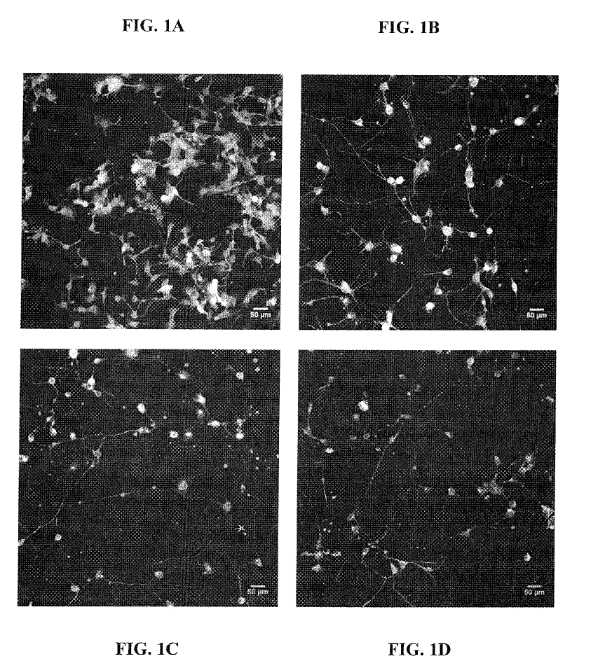

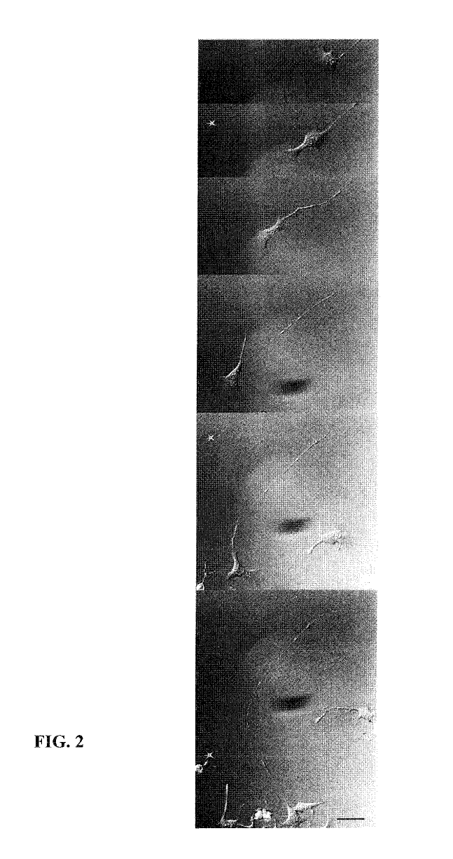

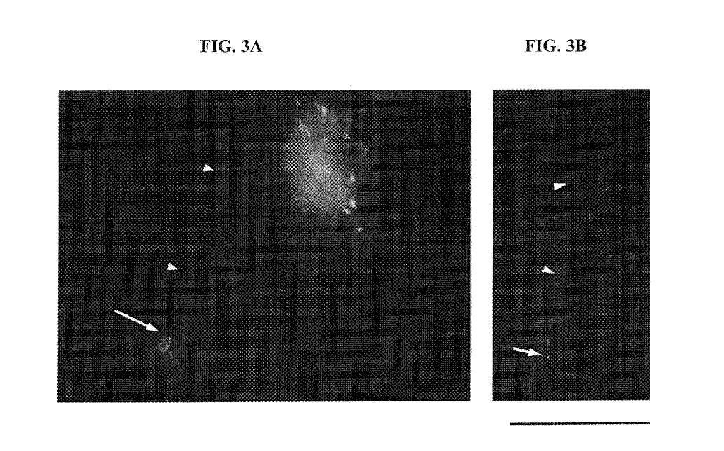

Isoquercitrin was shown to increase the length of neurite/cell and this effect is augmented by inhibitors of RhoA and ROCK kinase. In FIGS. 1A-1D, the formation of abundant neurites is apparent with isoquercitrin stimulation (nuclei are stained blue with DAPI, actin cytoskeleton is marked red with phalloidin and synaptotagmin-1 is green by immunostaining). Synaptotagmin-1, that is a calcium sensitive protein involved in synaptic vesicle docking and fusion, is shown to be highly expressed in the isoquercitrin-induced neurites. Time lapse images of a single neurite being pulled out of the soma of a NG108-15 cell are shown in FIG. 2. The tail of the cell fails to retract in part due to the formation of focal adhesions in the neurite tail (evidenced by the expression of vinculin spots—FIGS. 3A-3B) and, as later shown, by a reduction in RhoA activity.

### Example 2

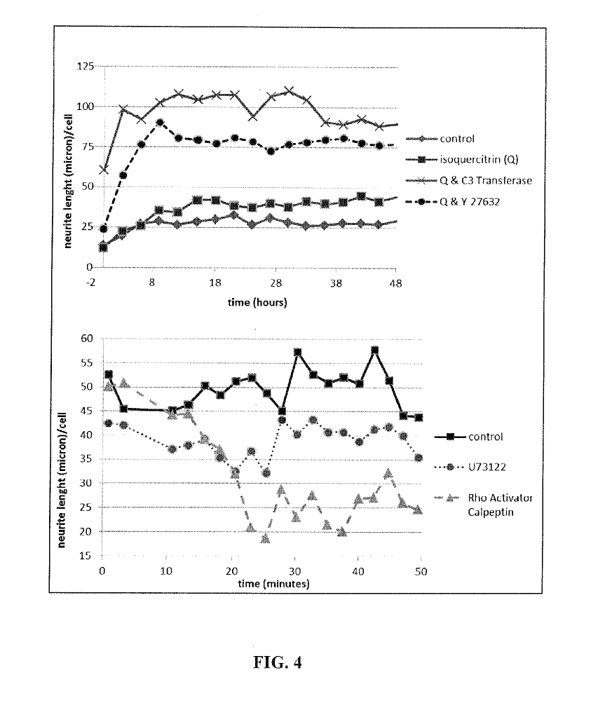

Segmentation of time-lapse images taken during 48 hrs of isoquercitrin incubation with/without Rho inhibitors all showed a significant increase in the neurite length/cell above control (FIG. 4).

### Example 3

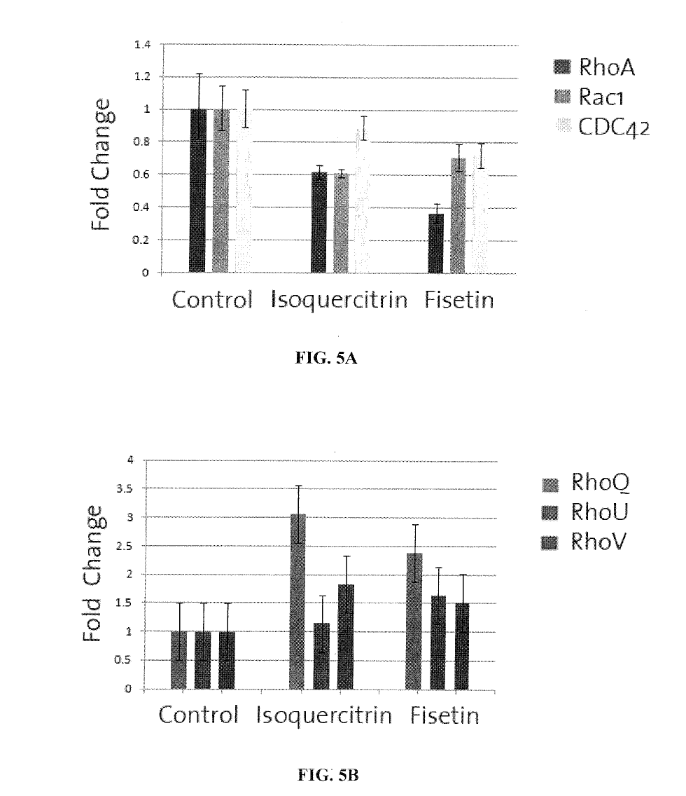

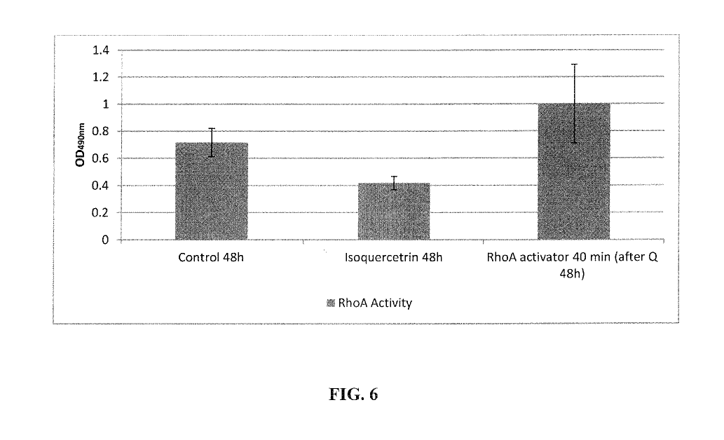

Effect of isoquercitrin on the gene expression of commonly studied RhoGTPases was examined. Rho GTPases are molecular switches which cycle between the GDP-bound and GTP-bound forms and have a potent influence on cell morphology as well as many other cellular functions. We found that the classical Rho GTPases, i.e. RhoA, Rac1 and CDC42, were down-regulated upon isoquercitrin stimulation (FIGS. 5A-5B). Furthermore, there was a consistent up-regulation of RhoQ, a RhoGTPase with a putative role in nerve regeneration. ELISA assay showed a downregulation of RhoA activation upon 48 hrs of isoquercitrin stimulation (FIG. 6).

### Example 4

Microarray analyses revealed global changes in gene expression due to the presence of 40 uM isoquercitrin and indicated that isoquercitrin induces neurodifferentiation as seen in Table 1, below:

TABLE I
Genes associates with neurogenesis and synapse formation
which were significantly upregulated by 40 uM isoquercitrin
treatment (Fold-change and p value)

| | | |
|---|---|---|
| GABA A receptor-associated protein-like 1 Gene | 2.361 | 0.02241 |
| [Source: MGI Symbol; Acc: MGI: 1914980] | | |
| synaptogyrin 1 Gene [Source: MGI | 2.130 | 0.0046 |
| Symbol; Acc: MGI: 1328323] | | |
| synaptotagmin I Gene [Source: MGI | 1.934 | 0.02136 |
| Symbol; Acc: MGI: 99667] | | |
| synaptotagmin XI Gene [Source: MGI | 1.913 | 0.0110 |
| Symbol; Acc: MGI: 1859547] | | |
| synaptotagmin IV Gene [Source: MGI | 1.825 | 0.08368 |
| Symbol; Acc: MGI: 101759] | | |
| glutamate receptor, ionotropic, N-methyl | 1.595 | 0.03916 |
| D-aspartate-associated protein 1 Gene | | |
| [MGI: 1913418] | | |
| cortactin binding protein 2 Gene [Source: MGI | 1.518 | 0.07128 |
| Symbol; Acc: MGI: 1353467] | | |
| synapsin II Gene [Source: MGI | 1.518 | 0.07174 |
| Symbol; Acc: MGI: 103020] | | |
| cholinergic receptor, nicotinic, alpha polypeptide | 1.438 | 0.04284 |
| 10 Gene [Source: MGI Symbol; Acc: MGI: 3609260] | | |
| protein kinase C and casein kinase substrate in | 1.398 | 0.05645 |
| neurons 1 Gene [Source: MGI | | |
| Symbol; Acc: MGI: 1345181] | | |
| synaptopodin 2-like Gene [Source: MGI | 1.393 | 0.003612 |
| Symbol; Acc: MGI: 1916010] | | |
| neural proliferation, differentiation and control | 1.385 | 0.03523 |
| gene 1 Gene [Source: MGI | | |
| Symbol; Acc: MGI: 1099802] | | |
| neurotrophin 5 Gene [Source: MGI | 1.325 | 0.04919 |
| Symbol; Acc: MGI: 97381] | | |
| reticulon 3 Gene [Source: MGI | 1.316 | 0.06292 |
| Symbol; Acc: MGI: 1339970] | | |
| calcium and integrin binding 1 (calmyrin) Gene | 1.297 | 0.02868 |
| [Source: MGI Symbol; Acc: MGI: 1344418] | | |
| synapsin I Gene [Source: MGI | 1.287 | 0.05145 |
| Symbol; Acc: MGI: 98460] | | |
| neuroligin 2 Gene [Source: MGI | 1.266 | 0.05061 |
| Symbol; Acc: MGI: 2681835] | | |
| dopamine receptor 4 Gene [Source: MGI | 1.248 | 0.02647 |
| Symbol; Acc: MGI: 94926] | | |

### Summary Observations

NG108-15 neuroblastoma-glioma hybridoma cells and the ND7/23 neural cell line were incubated in the presence of 15 flavonoids, either aglycons or glycosides. Among them, fisetin, kaempferol and quercetin-3-β-D-glycoside (isoquercitrin) all produced significant neurites, however, isoquercitrin-induced neurite formation was the most extensive and this was the only flavonoid to function at low cell densities. The neurite networks also persisted the longest (up to 4 days of culture) in comparison to the other substances tested, which induced apoptosis after 24 hrs. Interestingly, primary stem cells, enteric neural stem cells and fibroblasts were also induced to develop an extended neuronal phenotype following isoquecitrin treatment. These findings suggest that multiple cell types possess the adhesive and migratory machinery to produce neurite-like structures upon proper pharmacologic stimulation. Microarray data of flavonoid-stimulated NG108 cells showed a significant up-regulation of genes associated with neurotransmitter receptors (dopamine receptor 4 and GABA A receptor-associated protein-like 1), synapses (synaptogyrin, synaptotagmin, synaptopodin, synapsin) and focal adhesion proteins (paxillin and zyxin) among others. In conclusion, the flavonol isoquercitrin has a potent effect on neurodifferentiation, neuroregeneration and synapse protein expression and can be used to treat multiple neurologic and cognitive disorders.

Using the teachings provided herein, one skilled in the art will be able to determine suitable treatment protocols for administering the pharmaceutical compositions of the present invention. Further, conventional animal models may be used to determine therapeutic utility of compositions comprising flavonol glucosides in accordance with the present invention.

Also, stereoisomers of flavonol glucosides of Formula I exhibiting therapeutic activity are suitable for use in the present invention, and therefore are within the scope of the present invention.

The invention has been described with references to various and specific embodiments and techniques. It will be understood, however, that modifications of such embodiments and techniques can be made without departing from the spirit and scope of the invention. For example, the flavonol glycosides of the present invention may be coated on or supported by a matrix material, e.g. a scaffolding, porous sheet, wire or braid formed of a biocompatible material such as polymeric material or a metal or alloy, and implanted in the body as described, for example in an article by Yarlagadda, et al. entitled Recent Advances and Current Developments in Tissue Scaffolding, published in Bio-Medical Materials and Engineering 15(3), pp. 159-177 (2005). See also, U.S. Pat. Nos. 4,983,184, and 5,030,233. U.S. Published Applications Nos. U.S. 2009/0187258-A1, U.S. 2009/0228021-A1 and U.S. 2011/0137419-A 1, and PCT Publication No. WO2008/063526-A1, the contents of which are incorporated herein in their entireties, by reference.

### REFERENCES

[1] Y. Sagara, J. Vanhnasy, P. Maher, Journal of Neurochemistry 2004, 90, 1144.
[2] I. Baek, S. Jeon, M. Song, E. Yang, U. Sohn, I. Kim, Korean J Physiol Pharmacol 2009, 13, 201.
[3] A. J. Ridley, A. Hall, Cell 1992, 70, 389.
[4] D. J. Webb, J. T. Parsons, A. F. Horwitz, Nat Cell Biol 2002, 4, E97.
[5] M. Larsen, M. L. Tremblay, K. M. Yamada, Nat Rev Mol Cell Biol 2003, 4, 700.
[6] A. V. Somlyo, D. Bradshaw, S. Ramos, C. Murphy, C. E, Myers, A. P. Somlyo, Biochemical and Biophysical Research Communications 2000, 269, 652.
[7] R. M. Scaife, D. Job, W. Y. Langdon, Mol. Biol. Cell 2003, 14, 4605.
[8] S. Y. Gao, C. Y. Li, J. Chen, L. Pan, S. Saito, T. Terashita, K. Saito, K. Miyawaki, K. Shigemoto, K. Mominoki, S. Matsuda, N. Kobayashi, Nephron Experimental Nephrology 2004, 97, e49.
[9] C. L. Sayas, M. T. Moreno-Flores, J. s. Avila, F. Wandosell, Journal of Biological Chemistry 1999, 274, 37046.
[10] F. Postma, K. Jalink, T. Gengeveld, W. Moolenaar, EMBO Journal 1996, 15, 2388.
[11] M. Hirose, T. Ishizaki, N. Watanabe, M. Uehata, O. Kranenburg, W. H. Moolenaar, F. Matsumura, M. Maekawa, H. Bito, S. Narumiya, The Journal of Cell Biology 1998, 141, 1625.
[12] T. Minase, T. Ishima, K. Itoh, K. Hashimoto, European Journal of Pharmacology, 648, 67.
[13] R. Busca, C. Bertolotto, P. Abbe, W. Englaro, T. Ishizaki, S. Narumiya, P. Boquet, J.-P. Ortonne, R. Ballotti, Mol. Biol. Cell 1998, 9, 1367.
[14] T. Kubo, T. Yamashita, Recent Pat CNS Drug Discov 2007, 2, 173.

We claim:

1. A method to stimulate neurogenesis in cells and tissues in living humans and animals, comprising administering a therapeutically effective amount of a flavonol glycoside to a said person or animal.

2. The method according to claim 1, wherein the flavonol glycoside is administered to treat spinal cord or peripheral nerve injuries, stroke, neurological and cognitive disorders, memory loss or dementia suffered by said person or animal.

3. The method according to claim 1, wherein the cells are neurons, neural progenitor cells, adult stem cells, embryonic stem cells, induced pluripotent cells, connective tissue cells.

4. The method according to claim 1, wherein the flavonol glycoside has the following Formula I 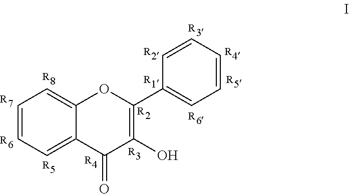
wherein each of R₅, R₆, R₇, R₈, R_{2′}, R_{3′}, R_{4′} and R_{5′} is independently H, OH or OCH₃, and each of R₃, R₇, R₈ and R_{4′} is independently a hydrogen atom or a monosaccharide bound to its respective ring structure by an O—, S—, N— or C— glycosidine bond through one of R₃, R₇, R₈ or R_{4′}.

5. The method of claim 4, wherein the monosaccharide is selected from the group consisting of a glucoside, a glucorhamnoside, a galactoside, a glucuronide, and a xyloside.

6. The method according to claim 1 where the flavonol glycoside is applied via injection directly in the site of neural injury.

7. The method of claim 1, wherein the flavonoid glycoside is administered via inhalation or orally.

8. The method of claim 1, wherein the flavonoid glycoside is administered via a suppository or by topical application.

9. The method of claim 1, wherein the flavonoid glycoside is administered in the form of eyedrops, or parenterally by subcutaneous injection, intravenously, intramuscularly or intrasternally.

10. The method according to claim 1, where the flavonoid glycoside is applied in in conjunction with a Rho inhibitor.

11. The method according to claim 10, wherein the Rho inhibitor is Y-27632, Fasudil, Exoenzyme C3 Transferase.

12. The method according to claim 1, wherein the flavonoid glycoside reduces the activation state of RhoA, Rac1 and/or CDC42.

13. The method according to claim 1, wherein the flavonoid glycoside increases the activation state of RhoQ, RhoU and/or RhoV.

14. The method according to claim 3, including the step of manipulating the cells ex vivo before transplantation into the human or animal body through incubation with the flavonol glycoside.

15. The method according to claim 3, including the step of manipulating the cells through a flavonol glycoside-loaded drug delivery system following transplantation.

16. The method according to claim 1, wherein said therapeutically effective amount is from about 0.05 mg to 500 mg per day, or on an as-needed basis.

17. The method according to claim 1, wherein the flavonol glycoside is coated on or supported on a matrix formed of a biocompatible material implanted into said person or animal.

18. The method according to claim 17, wherein the flavonol glycoside is coated or supported on said matrix before the matrix is implanted into said person or animal.

19. The method according to claim 17, wherein the flavonol glycoside is coated or supported on said matrix after the matrix is implanted into said person or animal.

20. A pharmaceutical composition comprising a therapeutically effective amount of a flavonol glycoside of the Formula I 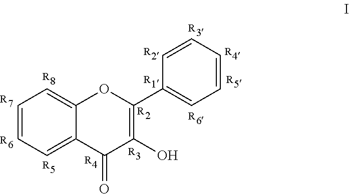
wherein each of R₅, R₆, R₇, R₈, R_{2′}, R_{3′}, R_{4′} and R_{5′} is independently H, OH or OCH₃, and each of R₃, R₇, R₈ and R_{4′} is independently a hydrogen atom or a monosaccharide bound to its respective ring structure by an O—, S—, N— or C— glycosidine bond through one of R₃, R₇, R₈ or R_{4′}.

21. The composition of claim 20, wherein the monosaccharide is selected from the group consisting of a glucoside, a glucorhamnoside, a galactoside, a glucuronide, and a xyloside.

22. The composition according to claim 20, where the composition also includes a Rho inhibitor.

23. The composition according to claim 20, wherein the Rho inhibitor is Y-27632, Fasudil, Exoenzyme C3 Transferase.

24. An implant for implanting into a person or animal, to stimulate neurogenisis in cells and tissues, comprising a matrix formed of a biocompatible material coated with or supporting a flavonol glycoside of the Formula I 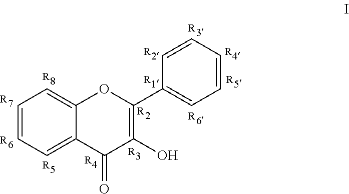
wherein each of R₅, R₆, R₇, R₈, R_{2′}, R_{3′}, R_{4′} and R_{5′} is independently H, OH or OCH₃, and each of R₃, R₇, R₈ and R_{4′} is independently a hydrogen atom or a monosaccharide bound to its respective ring structure by an O—, S—, N— or C— glycosidine bond through one of R₃, R₇, R₈ or R_{4′}.

25. The implant of claim 24, wherein the monosaccharide is selected from the group consisting of a glucoside, a glucorhamnoside, a galactoside, a glucuronide, and a xyloside.

26. The implant according to claim 24, where the composition also includes a Rho inhibitor.

27. The implant according to claim 24, wherein the Rho inhibitor is Y-27632, Fasudil, Exoenzyme C3 Transferase.
